(19) United States
(12) Patent Application Publication
(10) Pub. No.: US 2018/0099914 A1
(43) Pub. Date: Apr. 12, 2018

(54) RADIAL ELECTRO-MAGNETIC SYSTEM FOR THE CONVERSION OF SMALL HYDROCARBON MOLECULES TO LARGER HYDROCARBON MOLECULES USING A ROTATIONAL CHEMICAL REACTOR/SEPARATOR CHAMBER

(71) Applicant: Thomas Allen Hayes, Soldotna, AK (US)

(72) Inventor: Thomas Allen Hayes, Soldotna, AK (US)

(21) Appl. No.: 15/729,898

(22) Filed: Oct. 11, 2017

Related U.S. Application Data
(60) Provisional application No. 62/406,607, filed on Oct. 11, 2016.

(51) Int. Cl. C07C 2/80; B04B 5/10; C01B 3/34

(52) U.S. Cl. CPC C07C 2/80; C01B 3/342; B04B 5/10

(57) ABSTRACT

A system and a method are provided for an axial flow through chemical reactor that provides for the separation of hydrogen from a hydrocarbon feedstock and to form longer chain hydrocarbon molecules. The system consists of a radial magnetic field and an axial electric field in a cylindrical device, and a method of exciting flow through gas molecules by means of Lorentz Force to cause centrifugal force on the gas stream in the radial direction, inducing high molecular sheer in the rotating gas stream causes hydrogen to be removed from the rotating gas column, high molecular density forces radical hydrocarbon molecules to combine in the absence of Hydrogen.

### PRIORITY

This Application claims priority to U.S. provisional application No. 62/406,607, filed on Oct. 11, 2016, the entirety of which is incorporated herein by reference.

### TECHNICAL FIELD

Provided is a radial electro-magnetic system, apparatus and method for converting small hydrocarbon molecules to larger hydrocarbon molecules.

### BACKGROUND

Initial designs of an electromagnetic centrifuge for the separation of hydrogen from natural gas feedstock to form hydrocarbon radicals use a magnetic field in the axial direction and an electric field in the radial direction to induce Lorentz forces on a plurality of charged gas particles. The Lorentz forces cause the gas to rotate in a circular chamber without any mechanical motion sufficient enough to cause a high molecular density layer to form near the outer chamber circumference. The high velocity and molecular density causes hydrogen to be fractured from the feedstock gas molecule, allowing it to be separated from the chemical reaction and promotes the molecular re-combination of hydrocarbon radicals. Concepts of controlled turbulence, temperatures pressures, electron densities and profiles by RF, microwaves, UV and rotational frequency are taken into account. The entire apparatus can be used as a new type of chemical reactor. This design is considered a Radial Flow centrifuge. (Wong 201501580008)

In previous embodiments, issues arose from a radial flow centrifuge where the electrical conductivity from inner to outer electrode must conduct through the high density gas layer near the outer electrode. This layer is where the molecular velocities and densities are sufficient to cause the fracturing of the feedstock gas into hydrogen and hydrocarbon radicals and promotes the re-combination of these radicals into heavier gases and liquids. The electrical conduction path through this high density gas layer causes these heavier molecules to re-fracture into smaller hydrocarbon radical, reversing the process and limiting the effectiveness of the apparatus.

The axial magnetic field causes very high attractive forces pulling the two magnetic plates together. This causes a serviceability problem. Access to the inner electrode and radiofrequency (RF) electrode can only be performed by separating the magnetic plates and must be done with additional mechanical devices.

### SUMMARY

Provided is a method of performing an in-flow conversion of short chain hydrocarbons to larger chain hydrocarbon molecules. The method includes the following steps: providing a feedstock gas into the intake; generating an electric field in the direction of gas flow; injecting energy to partially ionize the gas mixture; generating a radial magnetic field perpendicular to the axial electric field; inducing a radial force on the flowing ionized gas column; inducing molecular shear to separate hydrogen from the ionized feedstock gas to produce hydrocarbon radicals; inducing molecular recombination of atomic hydrogen into H₂; inducing molecular recombination of the hydrocarbon radicals into larger molecules; inducing a controlled chemical reaction chain using a catalyst; inducing a molecular recombination with another reactant feedstock to produce larger molecules with both feedstock and reactant molecular components; producing a liquid hydrocarbon/reactant molecule; recovering the liquid hydrocarbon/reactant molecule from the feedstock exhaust; and, controlling recirculation of the un-reacted exhaust gases back to the intake.

According to further embodiments, the step of injecting includes using radiofrequency (RF) energy.

According to further embodiments, the radial magnetic field is created within a device which uses an outer ring of permanent magnets, with or without an inner ring of permanent magnets.

According to further embodiments, the step of generating the magnetic field includes the use of an alternating current (AC) magnet array.

According to further embodiments, the radial magnetic field is created using an alternating current (AC) Magnetic Coil Array and an inner magnetic conduction ring that produces an alternating radial magnetic field.

According to further embodiments, the electrodes are segmented into element pairs that conduct current when each of the peak alternating current (AC) magnetic fields are aligned with each electrode segment.

According to further embodiments, the step of generating an electric field comprises generating an offset alternating current (AC) electric field to induce an axial force vector in conjunction with the radial force vector.

According to further embodiments, the electric field is generated by supplying voltage to at least one electrode pair through a resonant LC transformer to compensate for the negative plasma voltage/current relationship, wherein the electrode pair comprises a positive electrode terminal and a negative electrode terminal.

According to further embodiments, wherein an electrode potential can be created using a high voltage phase control for switching power supply for each electrode pair.

According to further embodiments, an alternating current (AC) electrode potential can be created from a combination of magnetic windings on an alternating current (AC) magnetic coil array.

According to further embodiments, spinning gas caused by the radial force interfaces with angled radial and axial compressor blades causing a pressure increase in the output stage of the centrifuge.

According to further embodiments, a catalyst and/or secondary reactant gaseous feedstock compounds are added to improve molecular species reformation and conversion rates.

Also provided is an electro-magnetic vertical axis centrifuge. The electro-magnetic vertical axis centrifuge includes the following components: an upper manifold and a lower manifold connected respectively to an upper and a lower lid; an inner chamber wall and an outer chamber wall, wherein the outer chamber wall is sealed against the upper and lower lids with an outer pressure seal and an inner vacuum seal, wherein the inner chamber wall is supported by an upper support assembly and a lower support assembly; a magnetic flux return core, wherein the magnetic flux return core is supported by an upper support assembly and a lower support assembly; a plurality of magnetic induction cores which form a magnet ring; windings through the magnetic induction cores; windings from an induction core adjacent to the magnetic induction core; a common induction core leg; an upper electrode segment and a lower electrode segment and a radiofrequency (RF) electrode; a feedstock port, a hydrogen port and a reactant port and a syngas port, wherein the hydrogen gas port and syngas port are connected to vacuum pumps to provide gas flow; and radial compressor blades positioned upstream from the hydrogen port.

According to further embodiments, the magnetic induction core, the adjacent induction core and associated windings are clamped together with a core clamp late and heatsink assembly.

According to further embodiments, feedstock gas is introduced to the feedstock port to allow gas to flow through the upper manifold and past the RF electrode to ionize the gas.

According to further embodiments, an electrical current is applied between the upper electrode segment and the lower electrode segment to provide an electrically conductive gap within a partial vacuum containing the ionized feedstock gas.

According to further embodiments, the electrical current between the upper electrode segment and the lower electrode segment creates a vertical current path which intersects a horizontal magnetic path created between the magnetic induction cores and the magnetic flux return core to produce a perpendicular electric and magnetic field.

According to further embodiments, the perpendicular electric and magnetic field produces a Lorentz Force that exerts a force on the ionized gas and causes it to rotate.

According to further embodiments, the ionized gas forms a high molecular boundary layer near the outer chamber wall.

According to further embodiments, relatively high molecular weight gases flow through the syngas port and wherein lighter molecular weight gases flow through radial compressor blades and are compressed prior to flowing through the hydrogen port.

### BRIEF DESCRIPTION OF THE DRAWINGS

FIG. 1 is a side cross-sectional plan view of an electro-magnetic vertical axis centrifuge

FIG. 2 is a top cross-sectional plan view of an electro-magnetic vertical axis centrifuge.

FIG. 3 is a top cross-sectional plan view of a permanent magnet vertical axis centrifuge.

FIG. 4 is a side cross-sectional plan view of a permanent magnet vertical axis centrifuge assembly.

FIG. 5 is a diagram of a resonant LC electrode power supply

FIG. 6 is a diagram of a control system

FIG. 7a is a diagram of a Lorentz force vector orientation.

FIG. 7b is a diagram of a vertical electric field.

FIG. 7c is a diagram of an offset electric field.

FIG. 7d is a diagram of a rotating gas column showing the Lorentz force vectors.

FIG. 7e is a diagram of chamber gas flow and electrical current.

FIG. 8 is a perspective view of a radiofrequency electrode.

### DETAILED DESCRIPTION

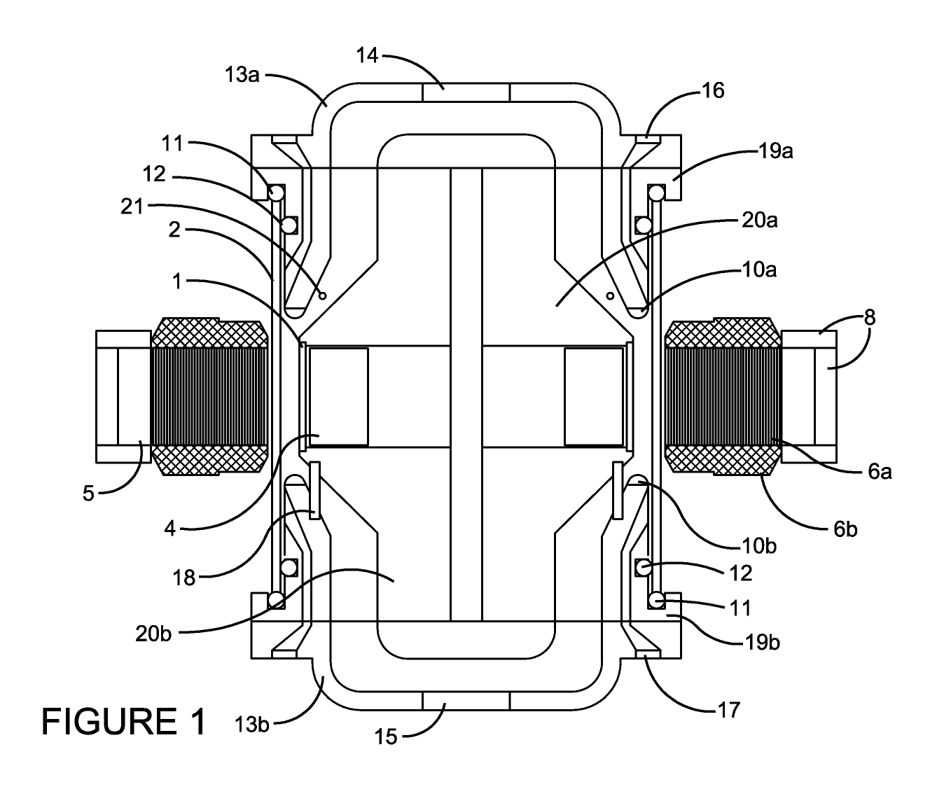

Referring now to the drawings wherein the showings are for purposes of illustrating embodiments of the invention and not for purposes of limiting the same, and wherein like reference numerals are understood to refer to like components, FIG. 1 shows one embodiment of a centrifuge, which according to the embodiment shown in FIG. 1, may be referred to as an electromagnetic vertical axis centrifuge. As shown in FIG. 1, centrifuge 100 comprises an upper manifold (13a) and lower (13b) manifold respectively connected to an upper lid (19a) and a lower lid (19b). The connection between each manifold and its respective lid may be accomplished by any means within sound engineering judgment, including, without limitation, mechanical fasteners, adhesives, welding and the like.

With continuing reference to FIG. 1, centrifuge (100) further comprises an outer chamber wall (2) that operatively seals against the upper lid (19a) and lower lid (19b). In one embodiment, a seal between the outer chamber wall (2) and upper and lower lids (19a, 19b) may be accomplished using one or both of an outer pressure seal (11), which may be a seal positioned between an end of the outer chamber wall and the respective lid (19a, 19b) and an inner vacuum seal (12), which may be a seal positioned between the inner face of the outer chamber wall and the respective lid. Whether one or more seals are used or other suitable means of creating a seal between the outer chamber wall and the respective lids are used, in some embodiments, the chamber created by the outer chamber wall, upper and lower lids and upper and lower manifolds may define a vacuum chamber, that is, a chamber on which a vacuum may be partially drawn. It will be understood, with reference to the Figures and description, that the lids and/or manifolds may be provided with one or more ports for the introduction or removal of gasses. These ports are described in further detail below. Inside of the vacuum chamber, there may be provided an upper support member (20a) and a lower support member (20b). The upper and lower support members may provide structural support for an inner chamber wall (1) and a magnetic flux return core (4).

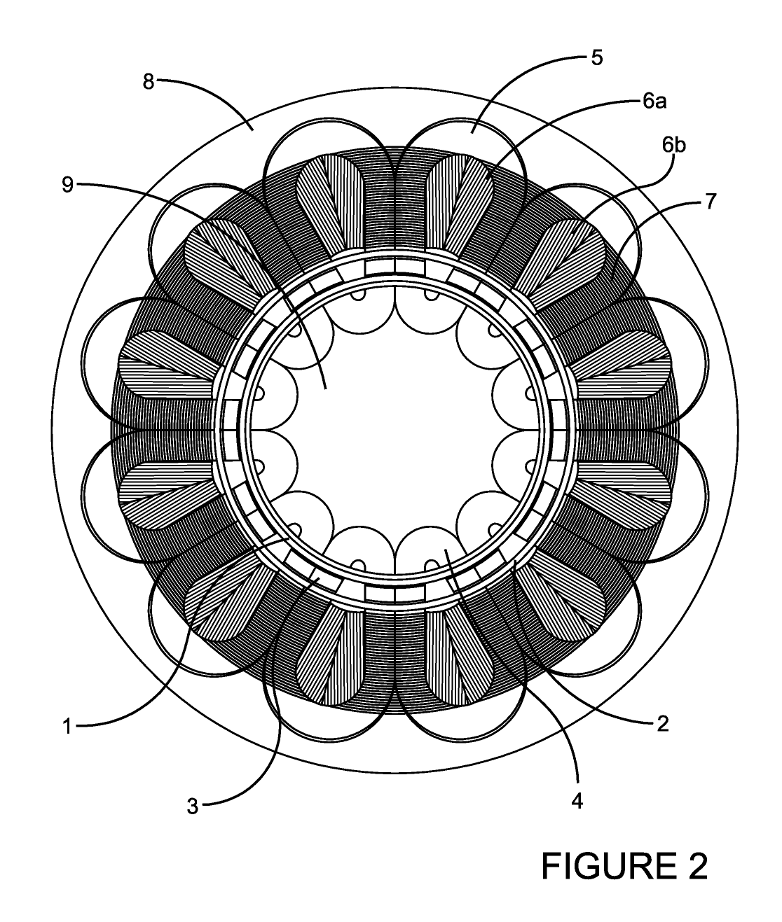

With reference to FIGS. 1 and 2, the centrifuge 100 may comprise an electromagnet ring array extending about the outer chamber wall and chamber. The electromagnetic ring array may comprise a plurality of magnetic induction cores (5) with a copper wire, or other suitable electrically conductive material, winding assembly that passes through a core center of one magnetic induction core (6a) and crosses over to the core center (6b) of an adjacent magnetic induction core. The electromagnet ring array comprising the magnetic induction cores and winding assembly may be clamped together with a core clamp plate and heatsink assembly (8).

With reference again to FIG. 1, a feedstock gas may be introduced to interior of the chamber through a feedstock port (14), which may, in one embodiment, be located on the upper manifold (13a). The feedstock port (14) may be adapted to allows the feedstock gas to flow through the upper manifold (13a) and past a radiofrequency (RF) electrode (21) located in the chamber. The RF electrode may be adapted, as described in detail below, to ionize the feedstock gas prior to the feedstock gas entering an electric field generated within the chamber. The electric field may be generated by applying an electrical voltage between an upper electrode segment (10a) and a lower electrode segment (10b), wherein the upper and lower electrode segments may be separated by a gap. This gap, when existing in a partial vacuum that may be drawn in the chamber and in the presence of the ionized feedstock gas is electrically conductive and allows current to flow between the upper and lower electrode segments. this ‘vertical’ current path intersects the ‘horizontal’ magnetic path created between the magnetic induction cores (5) and the magnetic flux return core (4). It will be understood that the terms ‘vertical’ and ‘horizontal’ are relative and not intended to be limiting. The perpendicular electric and magnetic fields produce a Lorentz Force that exerts a force on the ionized gas and causes it to rotate. This causes a high molecular boundary layer to form radially toward the inner surface of the outer chamber wall (2). A hydrogen port (15) and syngas port (17) may be connected to vacuum pumps to facilitate the flow of gas through the centrifuge chamber. The ionized rotating gas may, in some embodiments, be combined with a reactant gas stream, optionally in the presence of a catalyst, to form higher molecular weight reactant gas products. The vacuum and electromagnetic forces acting on the gas flow induce the gas to separate into higher and lower molecular weight portions and flow either through the syngas port (17) or the hydrogen port (15). In one embodiment, high molecular weight gases (that is, gases having a number average molecular weight of greater than the molecular weight of the original feedstock gas) flow through the syngas port (17) whereas lighter molecular weight gases, which may include one or more of hydrogen, unreacted feedstock gas and lighter MW fractured molecules may flow through radial compressor blades (18) where they are compressed prior to exiting the hydrogen port (15).

For purposes of the present invention, reference to “feedstock gasses” may include hydrocarbon based gasses, such as methane, ethane, propane, butane. Suitable gasses may be sourced or obtained from well flare gas capture, tank gas capture, bio-gas sources, or commercial natural gas.

FIG. 2 shows the interior cavity (9) of the chamber wherein is housed the radiofrequency (RF) resonant LC circuitry to provide electrical energy to the RF electrode (21). FIG. 2 shows the copper wire coils of the wire assembly that wind from one core center (6b), across to the adjacent core center (6b). Magnetic induction fields flow from the common induction core leg (7) across the gap between the inner (1) and outer (2) wall and are returned via the magnetic flux return core (4).

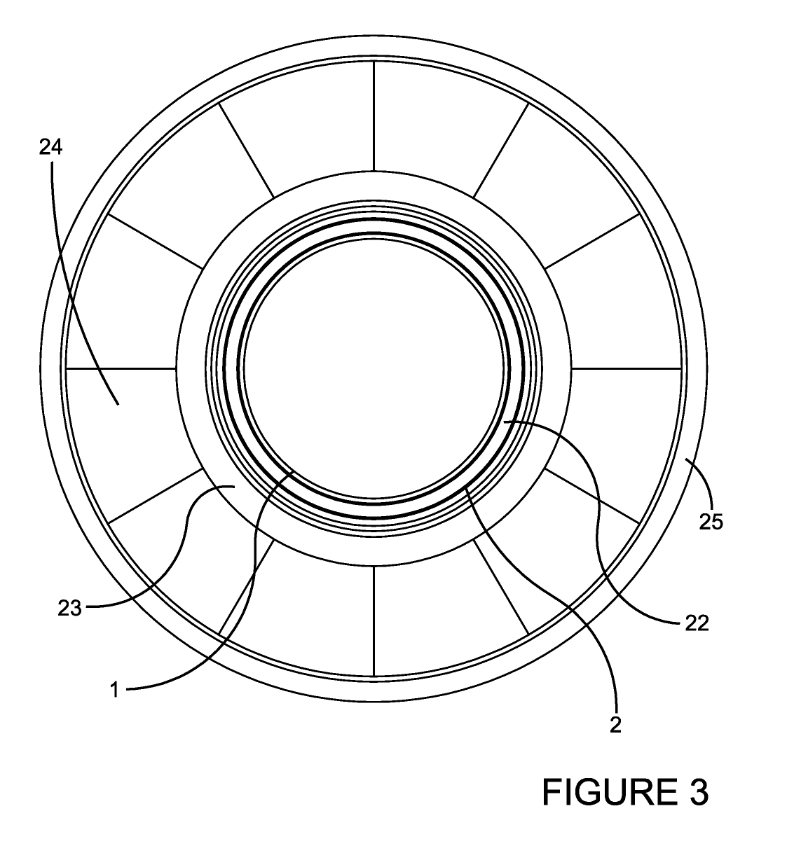

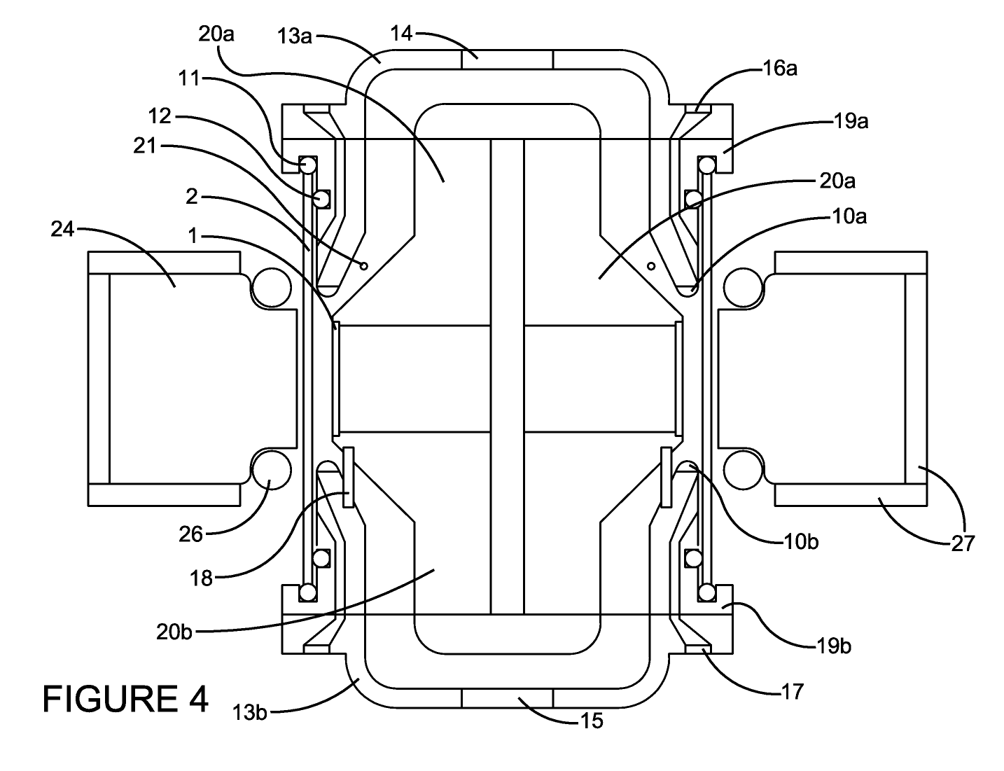

FIG. 3 depicts the magnet geometry in another embodiment of the centrifuge, more broadly shown in FIG. 4 and described below, which may be referred to as a permanent magnet vertical axis centrifuge. With reference to FIG. 3, a permanent magnet ring assembly comprises one or more magnet segments collectively forming a magnet ring about the outer wall of the centrifuge as depicted in FIG. 4. As in the electromagnet vertical axis centrifuge, introduced gas feedstock flows axially between the inner chamber wall (1) and the outer chamber wall (2). Adjacent the top and bottom of the chamber walls are respectively, an upper continuous electrode ring and a lower continuous electrode ring (shown generally as 10a). A DC current may be introduced to one of the upper or lower electrode rings. The respective electrodes may be arranged in such a manner that current flowing from the upper to lower electrodes intersects a magnetic field generated by the permanent magnet assembly (24) to induce Lorentz Forces perpendicular to the axial gas feedstock flow. One or more cooling coils (23) may be provided in proximity to the permanent magnet assembly to remove radiated heat generated from the permanent magnet ring assembly. A magnet retainer ring (25) may be employed to hold the permanent magnet segments (24) forming the magnet ring assembly together and to support the magnet ring assembly at equal distance from the outer chamber wall (2).

FIG. 4 depicts in further detail the an embodiment described as a permanent magnet vertical axis centrifuge wherein the centrifuge comprises a permanent magnet ring assembly comprising one or more magnet segments (24) rather than a magnet ring comprising a plurality of magnetic induction cores as described and shown in FIGS. 1 and 2. With continued reference to FIG. 4, the permanent magnet vertical axis centrifuge comprises a continuous upper electrode (10a) and a lower electrode (10b). The permanent magnet vertical axis centrifuge may comprise one or more cooling coils (26) providing sufficient cooling to limit the permanent magnet ring assembly's or individual magnet segments' temperature to below the Currie temperature. As with the electromagnetic vertical axis centrifuge, a feedstock gas may be introduced through a feedstock port (14), which gas may flow through the gap between the inner chamber wall (1) and the outer chamber wall (2) where it may be subjected to a continuous DC current with a continuous magnetic field. Also provided is RF electrode (21). As with the electromagnet vertical axis centrifuge, feedstock gas may be introduced into the chamber through port 14 and ionized in the presence of the RF electrode, at which time, one or more reactant gasses may be introduced into the ionized feedstock gas stream, optionally with one or more catalysts, to yield, inter alia, reactant gas products and hydrogen gas as previously described. This steady state current flow and magnetic field minimizes the boundary layer turbulence and aids in the separation of hydrogen.

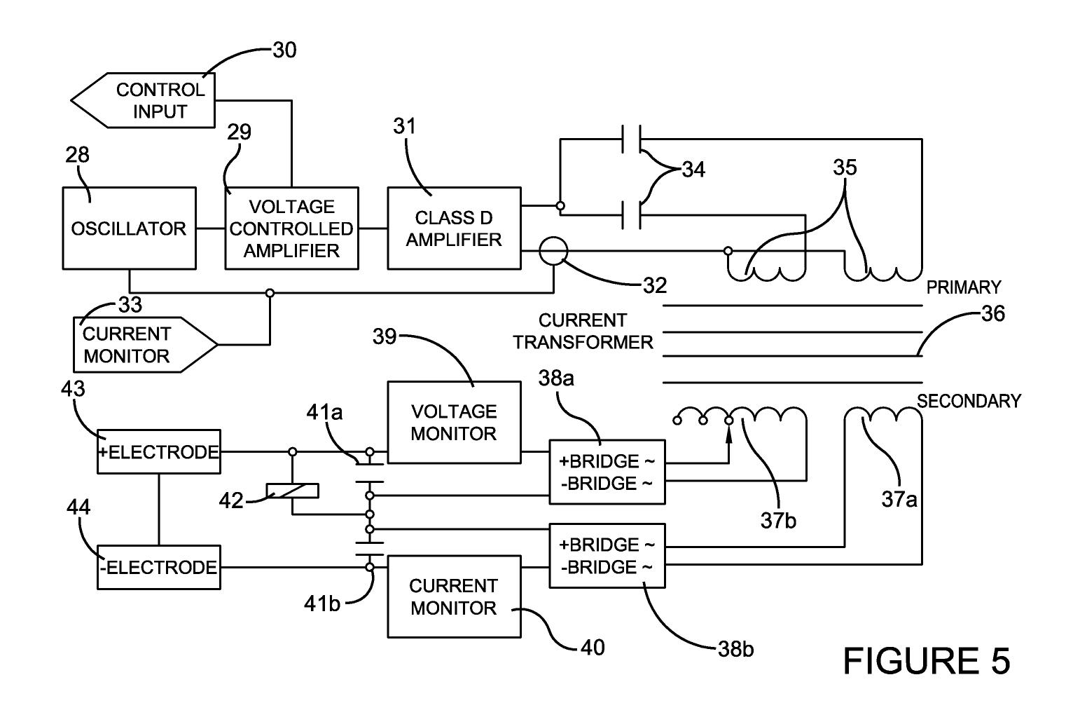

FIG. 5 depicts one embodiment of a resonant power supply system suitable for providing DC voltage to the upper and/or lower electrodes (10a, 10b). An oscillator (28) may be provided to initiate an LC resonance by starting at a fixed frequency close to the resonant frequency of the resonant inductor core (36) and the resonant capacitors (34). This resultant AC signal may be fed through a voltage controlled amplifier (29) that is modulated using a low voltage DC control signal via the control port (30) so as to cause the voltage controlled amplifiers output to vary based on the control input (30). A class D amplifier (31) amplifies this signal to provide a medium voltage high current ac voltage to the resonant LC circuit. A current transformer (32) measures the phase angle of the resonant LC circuit and causes the oscillator to adjust the frequency of operation to achieve a uniform power factor. An increase in voltage across the resonant primary inductor windings is a function of the circuit Q where Q=(reactive current/real current). Without a load on the secondary, the circuit Q will be greater than 100, causing the output of the resonant circuit to be 100 times that of the amplifiers output. A current monitor (33) allows the amplifiers current to be monitored by the control system. The fixed secondary winding (37a) produces a fixed AC voltage that is rectified to a DC voltage by lower bridge rectifier (38b) that charges the lower decoupling capacitor (41b). the adjustable secondary winding (37b) produces an adjustable ac voltage that is rectified by an upper bridge rectifier (38a) that charges the upper decoupling capacitor (41a) and allows the system to be tuned to varying load impedances. a voltage monitor (39) and a current monitor (40) allows the DC output voltage and current to be monitored by the control system. a voltage clamp (42) prevents the output from exceeding the upper (38a) and lower (38b) bridge rectifiers maximum voltage rating. the positive electrode terminal (43) and the negative electrode terminal (44) are connected to the upper and lower electrodes.

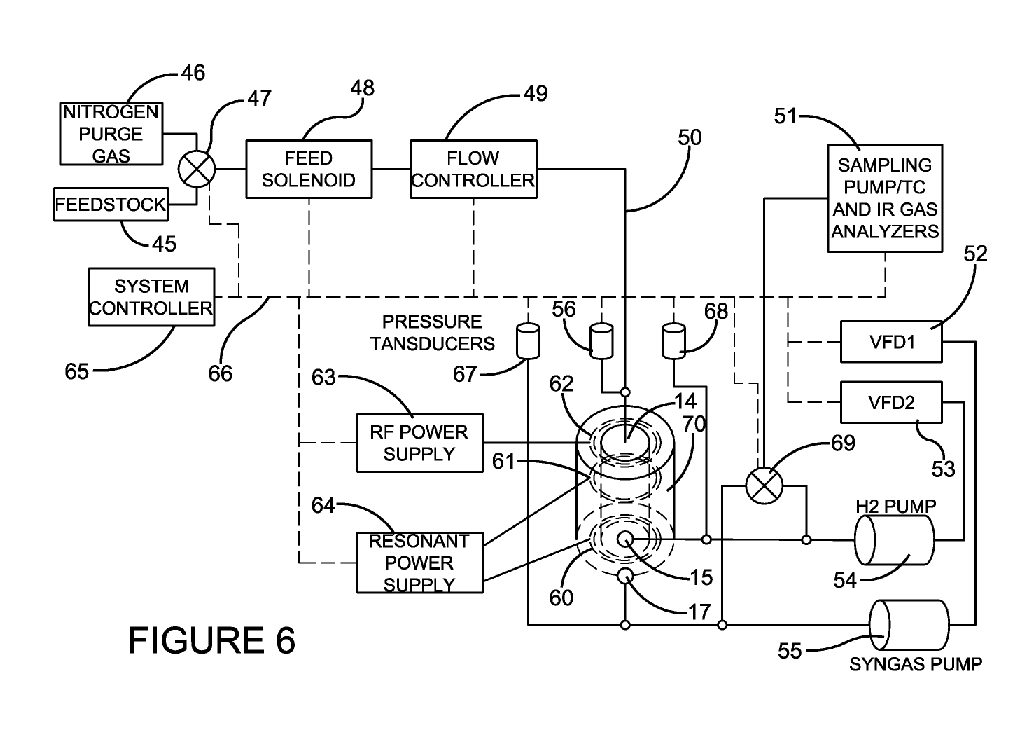

FIG. 6 depicts one embodiment of a control system (65) for a centrifuge according to the embodiments described and suitable to communicate to various monitor and control devices via a control and monitor buss. A feedstock gas port (45) may be fluidly connected to a raw gas feedstock source, and a purge gas port (46) may be connected to a nitrogen or other inert gas source. The controller may use a purge control valve (47) to fill the reactor chamber of the centrifuge with an inert gas prior to introducing the feedstock gas (or reactant gas) based on the controllers' programming. A feed solenoid (47) is controlled both by the controller and a manual safety switch to allow feedstock gas into the reactor chamber. The flow controller (49) allows the controller to vary the gas flow rates and provide low pressure gases to flow through a vacuum feed line (50) to the feedstock port (14) and enter the reactor for processing. A gas analyzer and pump (51) may be provided to sample either of the feedstock or reactant gases for analysis by the controller using sampling valve (69). Variable frequency drives, VFD1 (52) and VFD2 (52), may be employed to control a hydrogen pump (54) and a syngas pump (55) and are connected to the hydrogen port (15) and the syngas port (17) respectively by means of appropriate feed lines. A feed pressure transducer (56) may be connected to the feedstock port (14). A syngas port pressure transducer (68) may be connected to the syngas port (17) and a hydrogen port pressure transducer (67) may be connected to the hydrogen port (15) allowing the controller to monitor pressures at the outer perimeter of the reactor. The upper electrode (61) (10a) and lower electrode (60) (10b) may be connected to the resonant power supply (64) to provide DC voltage across the electrodes. The RF power supply (63) may be connected to the RF electrode (62).

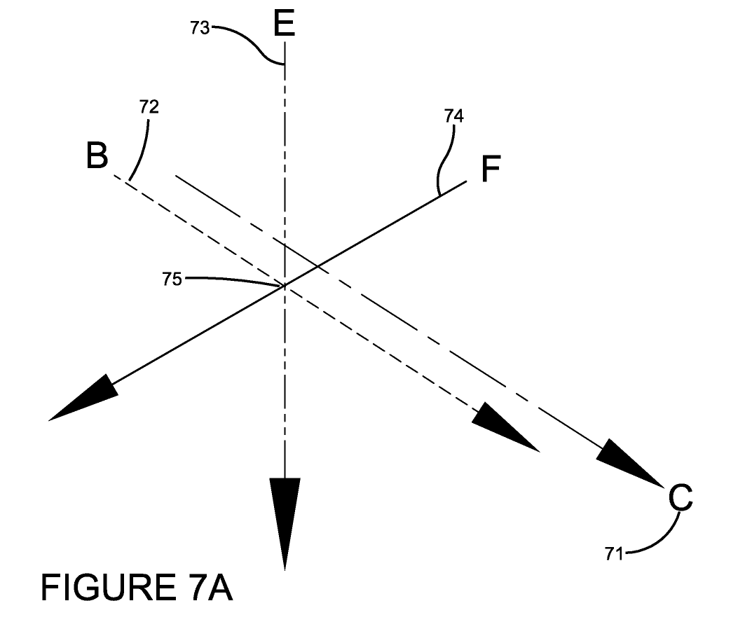

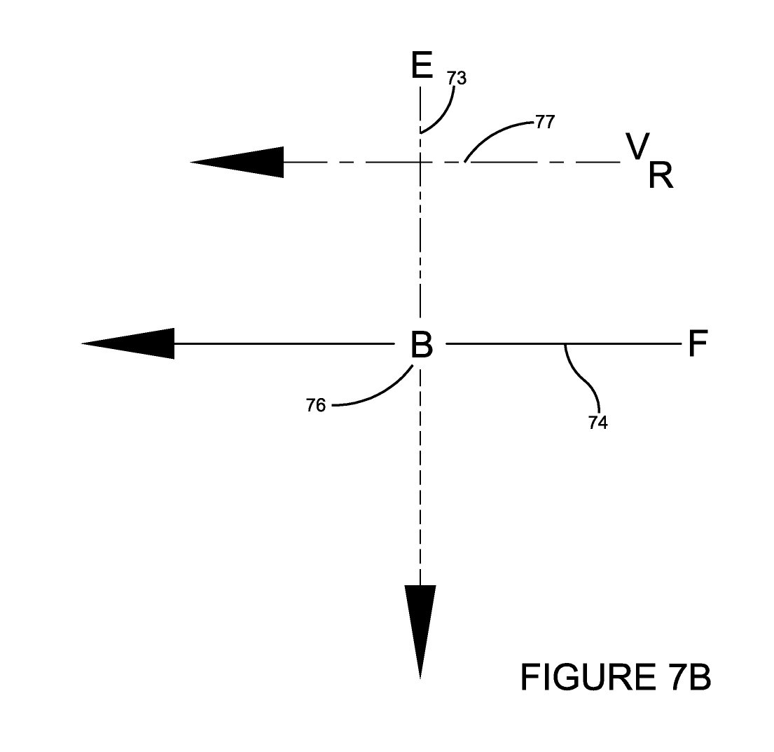

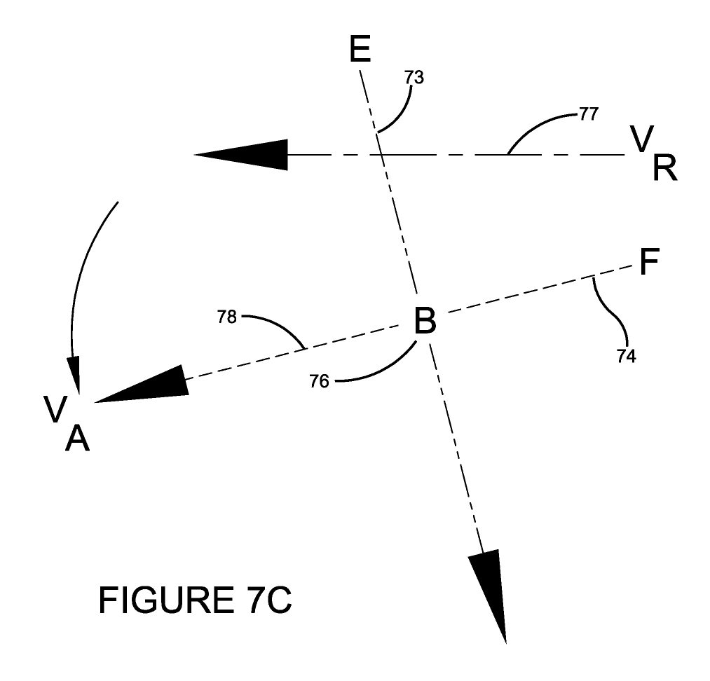

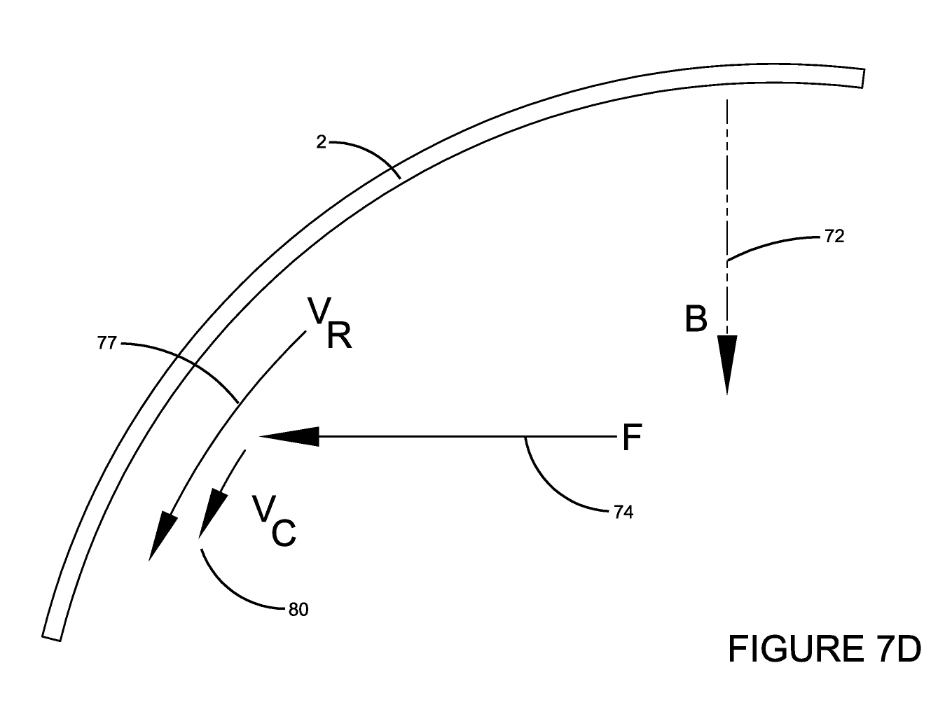

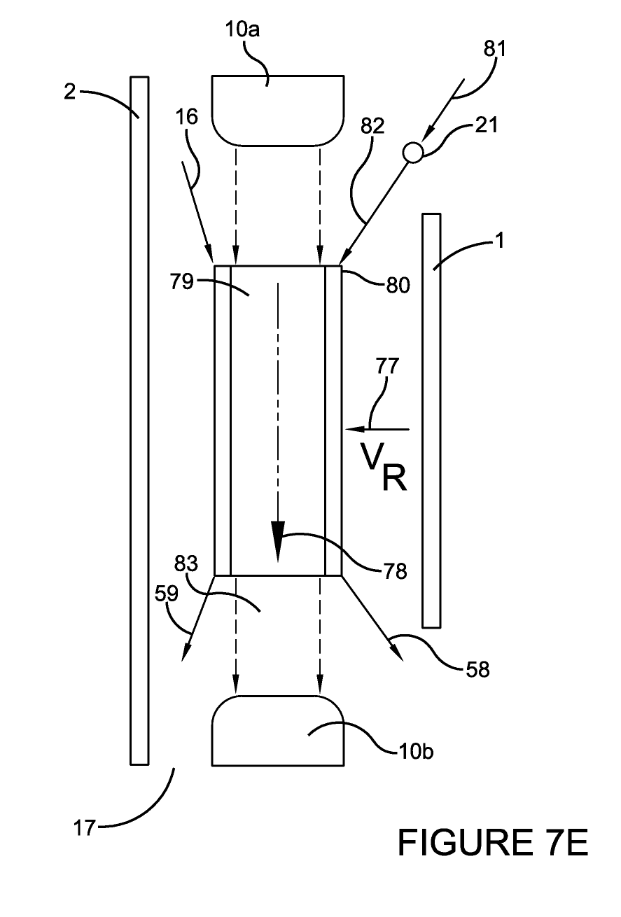

FIG. 7a depicts the Lorentz equation F=q(e+vb) where ‘F’ is the Lorentz Force Vector (74) exerted on ‘q’ Charged Particle (75) with an intersecting ‘β’ Magnetic Field (72) perpendicular to an ‘e’ Electric Field (73). This force causes a velocity increase perpendicular to the Center of Rotation (71).

FIG. 7b depicts a view from the center of rotation of the reactor with the 13′ magnetic force vector from center of radius (76) along with the vertical intersecting ‘e’ electric field (73) which results in the force ‘F’ Lorentz force vector (74). The rotating gas column contains both uncharged particles and ‘q’ charged particles (75) at rotating gas column Vr (77).

FIG. 7c depicts the offset electrode force vector for the segmented electrode pair current conduction path. The ‘e’ electric field (73) now angled causing an accompanying rotation of the ‘F’ Lorentz force vector (74). This produces a ‘Va” downward force vector to combine with the rotating gas column Vr (77).

FIG. 7d depicts the ‘W magnetic field (72) pointing inward towards the center of rotation creating the ‘F’ Lorentz force vector (74) forcing charged particles towards the outer chamber wall (2). The combined Vr rotating gas column (77) and the ‘F’ Lorentz force vector (74) intersect to create a Vc compression force (80) that forces the Vr rotating gas column (77) towards the outer chamber wall (2).

FIG. 7e depicts the system components and flow operation where the feedstock flow entry (81) flows past through the RF electrode (21) partially ionizing the gas feedstock (82) that then enters into a rotating gas column (79) and creates an impact to rotating gas column (80). This entry into the rotating gas column (79) by uncharged feedstock gases causes the molecular shear that creates both an ionized feedstock gas molecule and a free hydrogen ion. The rotating gas column (79) is contained between the inner chamber wall (1) and the outer chamber wall (2) and has electrical current flowing from the upper electrode ring (10a) and the lower electrode ring (10b). This creates the rotational force vector (77) that compresses the rotating gas column (79) into a high velocity, high molecular density zone close to the outer wall (2). A reactant gas may be introduced into the chamber through a reactant gas port (16). The reactant gas may merge with the rotating gas column (79). Suitable reactant gasses may include water vapor, ionic hydroxyls, heavier MW hydrocarbon gases, chlorine or fluorine gases, or neutral gases. Optionally, one or more catalysts may be introduced into the chamber to catalyze the reaction between the reactant gas and the ionized feedstock gas. Suitable catalysts may include platinum and other platinum group metal based metallic catalytic matrix and organic metal composites. The gas, now comprising a combination of unreacted reactant gas and ionized feedstock gas in addition to the reaction products of the reactant gas and the ionized gas, as well as hydrogen gas is drawn downward by vacuum and forced downwards by the Lorentz force axial (78) component and causes a higher molecular density at the molecular density separation zone where heavier molecules follow the syngas stream (59) and exit the system via the Syngas Port (17). The lighter hydrogen molecules follow the hydrogen stream (58).

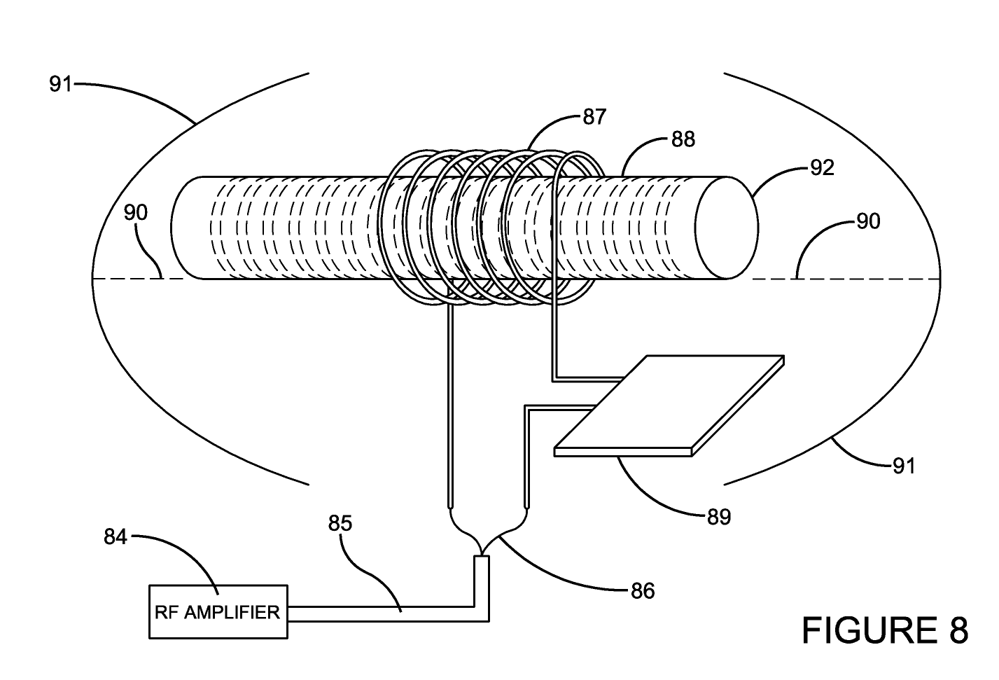

FIG. 8 depicts the RF electrode assembly where the RF amplifier (84) outputs an RF waveform through the RF cable (85). The RF coax cable splits (86) to drive the primary resonant air core inductor (87) that is tuned to match the radiofrequency (RF) capacitor (89) and created a radiofrequency (RF) voltage that the radiofrequency (RF) amplifier (84) amplifies into an output multiplied by the Q of the resonant inductor and capacitor. The high voltage radiofrequency (RF) signal across the primary resonant air core inductor (87) induces a voltage onto the secondary windings (88) which are wound around the secondary winding support tube (92). The output from the secondary windings (88) exit the RF electrode assembly through radiofrequency (RF) electrode leads (90) and connects to each of the dipole RF electrodes (91), (61) and (21). The radiofrequency RF coax cable splits (86), primary resonant air core inductor (87), secondary windings (88), RF capacitor (89), RF electrode leads (90) and dipole RF electrodes (91) are made of high temperature material and are housed inside of the upper support assembly (See FIG. 2—Reference No. 20a).

Having extensively described one or more embodiments of the centrifuge apparatus and control mechanism according to the present invention, now is described a method of employing the centrifuge apparatus in performing an in-flow conversion of short chain hydrocarbons to larger chain hydrocarbon molecules comprising the steps of providing a feedstock gas and a centrifuge as previously described. The method may comprise the further steps of introducing a flow of a feedstock gas through the intake port of a centrifuge. The method further comprises generating an electric field in the direction of gas flow through the centrifuge chamber; injecting energy, such as radiofrequency (RF) energy to partially ionize the gas mixture forming a flowing ionized gas column; generating a radial magnetic field perpendicular to the electric field. In one embodiment, the magnetic field may be generated using a permanent magnet ring assembly, which may comprise an outer ring of permanent magnet segments, with or without a cooperating inner ring of permanent magnet segments. In another embodiment, the magnetic field may be generated using an electro-magnet ring array.

The method may further comprise inducing a radial force on the flowing ionized gas column; inducing molecular shear to separate hydrogen from the ionized feedstock gas to produce hydrocarbon radicals; inducing molecular recombination of atomic hydrogen into H₂; inducing molecular recombination of the hydrocarbon radicals into larger molecules; inducing a controlled chemical reaction chain using a catalyst; inducing a molecular recombination with another reactant feedstock to produce larger molecules with both feedstock and reactant molecular components; producing a liquid hydrocarbon/reactant molecule; recovering the liquid hydrocarbon/reactant molecule from the feedstock exhaust; and, controlling recirculation of the un-reacted exhaust gases back to the intake.

What is claimed is:

1. A method of performing an in-flow conversion of short chain hydrocarbons to larger chain hydrocarbon molecules comprising:
providing a feedstock gas into the intake;
generating an electric field in the direction of gas flow;
injecting energy to partially ionize the gas mixture;
generating a radial magnetic field perpendicular to the axial electric field;
inducing a radial force on the flowing ionized gas column;
inducing molecular shear to separate hydrogen from the ionized feedstock gas to produce hydrocarbon radicals;
inducing molecular recombination of atomic hydrogen into H₂;
inducing molecular recombination of the hydrocarbon radicals into larger molecules;
inducing a controlled chemical reaction chain using a catalyst;
inducing a molecular recombination with another reactant feedstock to produce larger molecules with both feedstock and reactant molecular components;
producing a liquid hydrocarbon/reactant molecule;
recovering the liquid hydrocarbon/reactant molecule from the feedstock exhaust; and,
controlling recirculation of the un-reacted exhaust gases back to the intake.

2. The method as set forth in claim 1, wherein the step of injecting comprises using radiofrequency (RF) energy.

3. The method as set forth in claim 1, wherein the radial magnetic field is created within a device which uses an outer ring of permanent magnets, with or without an inner ring of permanent magnets.

4. The method as set forth in claim 1, wherein the step of generating the magnetic field includes the use of an alternating current (AC) magnet array.

5. The method as set forth in claim 1, wherein the radial magnetic field is created using an alternating current (AC) Magnetic Coil Array and an inner magnetic conduction ring that produces an alternating radial magnetic field.

6. The method as set forth in claim 1, wherein the electrodes are segmented into element pairs that conduct current when each of the peak alternating current (AC) magnetic fields are aligned with each electrode segment.

7. The method as set forth in claim 1, wherein the step of generating an electric field comprises generating an offset alternating current (AC) electric field to induce an axial force vector in conjunction with the radial force vector.

8. The method as set forth in claim 1, wherein the electric field is generated by supplying voltage to at least one electrode pair through a resonant LC transformer to compensate for the negative plasma voltage/current relationship, wherein the electrode pair comprises a positive electrode terminal and a negative electrode terminal.

9. The method as set forth in claim 8, wherein an electrode potential can be created using a high voltage phase control for switching power supply for each electrode pair.

10. The method as set forth in claim 9, wherein an alternating current (AC) electrode potential can be created from a combination of magnetic windings on an alternating current (AC) magnetic coil array.

11. The method as set forth in claim 1, wherein spinning gas caused by the radial force interfaces with angled radial and axial compressor blades causing a pressure increase in the output stage of the centrifuge.

12. The method as set forth in claim 1, wherein a catalyst and/or secondary reactant gaseous feedstock compounds are added to improve molecular species reformation and conversion rates.

13. An electro-magnetic vertical axis centrifuge comprising:
an upper manifold and a lower manifold connected respectively to an upper and a lower lid;
an inner chamber wall and an outer chamber wall, wherein the outer chamber wall is sealed against the upper and lower lids with an outer pressure seal and an inner vacuum seal, wherein the inner chamber wall is supported by an upper support assembly and a lower support assembly;
a magnetic flux return core, wherein the magnetic flux return core is supported by an upper support assembly and a lower support assembly;
a plurality of magnetic induction cores which form a magnet ring;
windings through the magnetic induction cores;
windings from an induction core adjacent to the magnetic induction core;
a common induction core leg;
an upper electrode segment and a lower electrode segment and a radiofrequency (RF) electrode;
a feedstock port, a hydrogen port and a reactant port and a syngas port, wherein the hydrogen gas port and syngas port are connected to vacuum pumps to provide gas flow; and
radial compressor blades positioned upstream from the hydrogen port.

14. The electro-magnetic vertical axis centrifuge of claim 13, wherein the magnetic induction core, the adjacent induction core and associated windings are clamped together with a core clamp late and heatsink assembly.

15. The electro-magnetic vertical axis centrifuge of claim 14, wherein feedstock gas is introduced to the feedstock port to allow gas to flow through the upper manifold and past the RF electrode to ionize the gas.

16. The electro-magnetic vertical axis centrifuge of claim 15, wherein an electrical current is applied between the upper electrode segment and the lower electrode segment to provide an electrically conductive gap within a partial vacuum containing the ionized feedstock gas.

17. The electro-magnetic vertical axis centrifuge of claim 16, wherein the electrical current between the upper electrode segment and the lower electrode segment creates a vertical current path which intersects a horizontal magnetic path created between the magnetic induction cores and the magnetic flux return core to produce a perpendicular electric and magnetic field.

18. The electro-magnetic vertical axis centrifuge of claim 17, wherein the perpendicular electric and magnetic field produces a Lorentz Force that exerts a force on the ionized gas and causes it to rotate.

19. The electro-magnetic vertical axis centrifuge of claim 18, wherein the ionized gas forms a high molecular boundary layer near the outer chamber wall.

20. The electro-magnetic vertical axis centrifuge of claim 19, wherein relatively high molecular weight gases flow through the syngas port and wherein lighter molecular weight gases flow through radial compressor blades and are compressed prior to flowing through the hydrogen port.
